(19) United States
(12) Patent Application Publication
(10) Pub. No.: US 2007/0000488 A1
(43) Pub. Date: Jan. 4, 2007

(54) Dosing device

(76) Inventors: Joachim Koerner, Uhldingen (DE); Michael Helmlinger, Radolfzell (DE); Holger Schuerle, Radolfzell (DE)

Correspondence Address: FLYNN THIEL BOUTELL & TANIS, P.C., 2026 RAMBLING ROAD, KALAMAZOO, MI, 49008-1631, US

(21) Appl. No.: 11/051,963

(22) Filed: Feb. 4, 2005

(30) Foreign Application Priority Data
Feb. 5, 2004 (DE) 102004006453.9

(51) Int. Cl. A61M 15/00; B65D 83/06

(52) U.S. Cl. 128/203.15

(57) ABSTRACT

1. Dosing device. 2.1 A dosing device for a medium, with a medium reservoir, with at least one medium channel for delivering the medium, and with at least one dispensing opening for dispensing the medium, is known. 2.2 According to the invention, the medium channel is assigned a control unit which comprises a measurement means for detecting an actual value of a stream of medium, a memory for desired values of the stream of medium, a comparator for comparison between actual values and desired values, and an actuating means for acting on the stream of medium as a function of evaluations performed by the comparator. 2.3 Use for dosing of medium.

The invention relates to a dosing device for a medium, with a medium reservoir, with at least one medium channel for delivering the medium, and with at least one dispensing opening for dispensing the medium.

Many different designs of dosing devices of this kind are known from the prior art. They are used for dosing and dispensing liquid or powder media in the cosmetics sector and in pharmacy. For this purpose, known dosing devices have a medium reservoir in which the medium to be dispensed is stored until the time of a dispensing operation. At least one medium channel is also provided which allows the medium to be delivered from the medium reservoir to a dispensing opening. The medium to be dispensed is forced through the dispensing opening under the effect of pressure and discharged into the environment of the dosing device.

The object of the invention is to make available a dosing device of the type mentioned at the outset which permits a more precise dispensing operation.

This object is achieved by the fact that the medium channel is assigned a control unit which comprises a measurement means for detecting an actual value of a stream of medium, a memory for desired values of the stream of medium, a comparator for comparison between actual values and desired values, and an actuating means for acting on the stream of medium as a function of evaluations performed by the comparator. A control units permits control of an actuating means by comparing and acting on actual values of a stream of medium on the basis of desired values for the stream of medium which are stored in a memory. The actual values of the stream of medium are acted upon according to the predetermined desired values. For this purpose, during the dispensing operation, a comparison between desired values and actual values is carried out permanently or at least temporarily, and a control signal resulting from the comparison is sent to the actuating means. In the actuating means, the control signal leads to a reduction or increase in the stream of medium. The actuating means can be configured in particular as a hydraulic or pneumatic control valve and/or as a pressure generator or pressure reducer.

The measurement means used can be, in particular, inductive, capacitive, optical or mechanical volumetric flow meters. Moreover, for the purpose of volumetric flow measurement, it is also possible to provide a differential pressure gauge with measuring diaphragm or an, in particular, optoelectrically scanned float. The memory for the desired values can be, in particular, an electrical, electronic or mechanical memory. The comparator can also entail electrical, electronic or mechanical comparing means. With the aid of the control unit, a predefined quantity of medium in particular can be dispensed, the predefined quantity being rigidly set or adjustable by the user.

In one embodiment of the invention, a dosing chamber provided on the medium channel at least temporarily receives a quantity of liquid and is assigned the at least one dispensing opening. The dosing chamber is provided with a vibration unit which is operatively connected at least to one boundary surface of the dosing chamber in order to cause this boundary surface to oscillate for the purpose of a dispensing operation. An output function unit connected to the vibration unit is provided for activating the vibration unit during an output period. A dosing chamber is defined by a plurality of boundary surfaces, the vibration unit being provided on at least one boundary surface. The vibration unit, which is designed in particular as a piezoelectric oscillator, can cause the boundary surface to oscillate by acting on it with an electrical voltage. For a dispensing operation, medium is introduced from an unpressurized medium reservoir into the dosing chamber in particular via a delivery means, such as a manually or electromechanically actuated pump means, by opening a valve on a pressurized medium reservoir or, with suitable configuration of the medium channel, by capillary forces. The dosing chamber is at atmospheric pressure. Such a configuration is suitable in particular for use in a microdosing device for high-precision dosing of very small quantities of liquid. One application lies in particular in the pharmaceutical sector.

At one boundary surface, the dosing chamber is assigned a dispensing opening through which the medium can pass only when it has been possible to overcome a flow resistance, defined in particular by the geometry of the dispensing opening, a surface characteristic of the boundary surface and the surface tension or viscosity of the medium. In order to be able to apply a suitable pressure to the medium in the dosing chamber, for the purpose of overcoming the flow resistance, the vibration unit is provided which creates pressure waves in the medium in the dosing chamber so that medium is dispensed at least temporarily from the dispensing opening. For activating the vibration unit, an output function unit is provided which outputs an in particular time-dependent electrical signal to the vibration unit.

In a further embodiment of the invention, the output function unit is coupled to the control unit for controlled output of medium. By coupling the output function unit to the control unit, synchronization can be produced between the actual value and desired value of the stream of medium and the activation of the vibration unit. In this way, the output function unit sends trigger signals to the vibration unit only for as long as is necessary to reach the desired value of the stream of medium or only for as long as the actual value of the stream of medium corresponds to the desired value.

In a further embodiment of the invention, the measurement means is provided in a storage chamber assigned to the dosing chamber. A storage chamber can in particular be coupled directly to the medium channel and/or can be connected to said medium channel via the dosing chamber. The storage chamber serves in particular to ensure a continuous volumetric flow without time-related bottlenecks during the dispensing of the medium. For this purpose, the storage chamber can in particular be provided as a capillary tube or cavity and be provided with a measurement means, in particular for determining a volume or a volumetric flow. Designing the measurement means as a volume meter is expedient when a volume of the storage chamber can be precisely determined. An arrangement as a volumetric flow meter is preferred if a geometry through which the medium to be dispensed flows can be precisely determined only in a defined portion of the storage chamber. Determining the volume dispensed from the storage chamber allows a conclusion to be drawn, directly or indirectly, concerning the quantity of medium dispensed overall from the dosing chamber and thus permits an exact determination of the quantity of medium dispensed from the dosing device during the dispensing operation.

In a further embodiment of the invention, the measurement means, seen in the direction of flow of the medium, is arranged downstream from the dispensing opening on the dosing chamber. As a result of pressure forces applied by delivery means and in particular by the vibration unit provided on the dosing chamber, the medium is subjected to a pressure and can be output into the environment of the dosing device counter to the flow resistance of the dispensing opening. In doing so, the medium adopts a clearly defined flow direction going from the medium reservoir via the delivery means, the dosing chamber and the dispensing opening into the environment. Arranging a measurement means downstream from the dispensing opening permits determination of the quantity of medium actually output into the environment through the dispensing opening.

In a further embodiment of the invention, the measurement means has an optical and/or capacitive and/or inductive sensor arrangement. For an optical sensor arrangement, which can be designed in the form of a light barrier or a reflectometer, a light signal is emitted from a light source, in particular almost parallel to a plane of extension of the boundary surface provided with the dispensing opening. Then, using a photosensitive sensor, it is possible to analyse the transmitted or the reflected light beam, as a result of which a measurement signal can be determined which gives information on the number and/or density of particles of liquid in a defined measurement stretch. Alternatively, or in addition, the measurement means can conceivably also be configured as a charge meter. In this case, dispensed particles of liquid are charged by an electrically biased boundary surface. In particular, each particle of liquid can be precisely assigned one charge unit or can be given a charge proportional to its size. By drawing up a charge balance, it is then possible to determine the number of charges lost and thus draw conclusions concerning the number of liquid particles that have been dispensed.

In a further embodiment of the invention, the medium reservoir is pressurized. Pressurizing the medium reservoir can be done in particular by means of a gaseous or liquid propellant provided in the medium reservoir and/or by mechanical pretensioning of a deformable or movable wall of the medium reservoir. The use of propellants, in particular of propellant gases such as carbon dioxide, permits a particularly simple mechanical configuration of the medium reservoir, since no movable parts are needed for pressurizing the medium. Mechanical pressurization, by contrast, permits reliable dispensing of the medium provided in the medium reservoir, even under widely different environmental conditions, for example in the event of temperature fluctuations. A pressurized medium reservoir also has the advantage that, when a dosing valve is opened, a substantially continuous stream of medium from the medium reservoir can be ensured.

The object of the invention is also achieved by the fact that a gas separator is provided in the medium channel and/or in the medium reservoir. A gas separator can be designed in particular as a semipermeable membrane which allows only clearly defined constituents of the medium to pass through into the environment, while other constituents of the medium are held back. The use of such a gas separator is particularly expedient when especially precise dosing is sought and when the medium has to be dispensed free from gas. Other components which are located in the medium reservoir and which would pose an impediment to volumetric flow measurement or volume measurement, cannot pass through the semipermeable membrane and are therefore not dispensed. These undesired constituents can be, in particular, gas particles which are dissolved in the medium and which can lead to measurement errors by the measurement means and/or to the formation of gas bubbles in the dosing chamber and, consequently, in particular to a reduction in the efficiency of dispensing and/or quality of dispensing.

In a further embodiment of the invention, a gas outlet is provided on the medium reservoir and/or on the gas separator. A gas outlet can in particular be designed as a hydrophobic membrane filter and serves to separate gaseous constituents from the liquid medium. The gas outlet can be switched either permanently for removal of gas from the medium or, as a function of control commands, only temporarily.

Further advantages and features of the invention will become evident from the claims and from the following description of preferred illustrative embodiments of the invention, as set out with reference to the drawings.

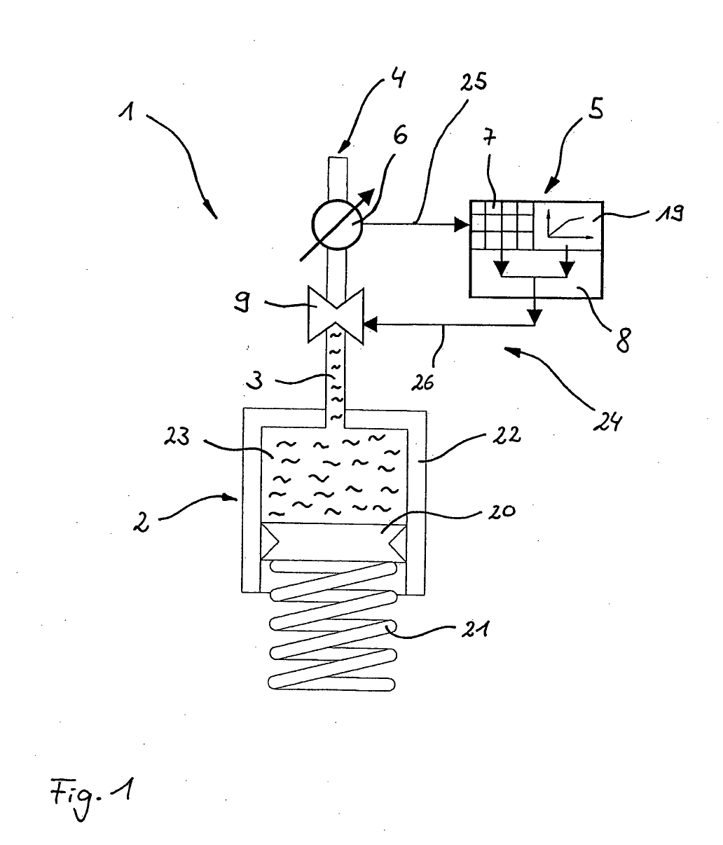

FIG. 1 shows, in a schematic representation, a dosing device comprising a control means,

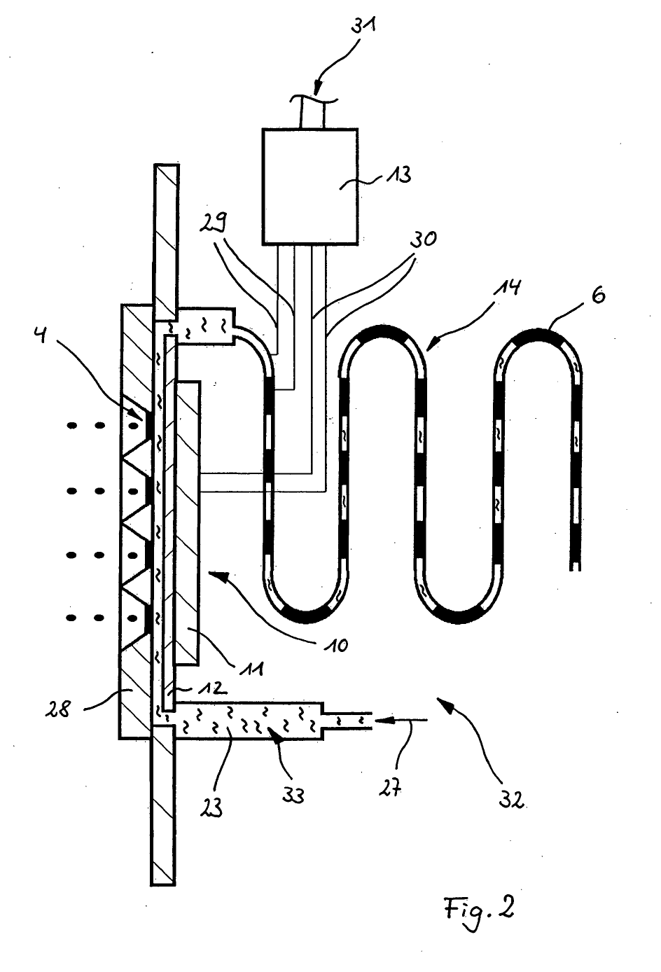

FIG. 2 shows, in a schematic representation, a dosing chamber of a dosing device, with an output function unit, a vibration unit and a measurement means,

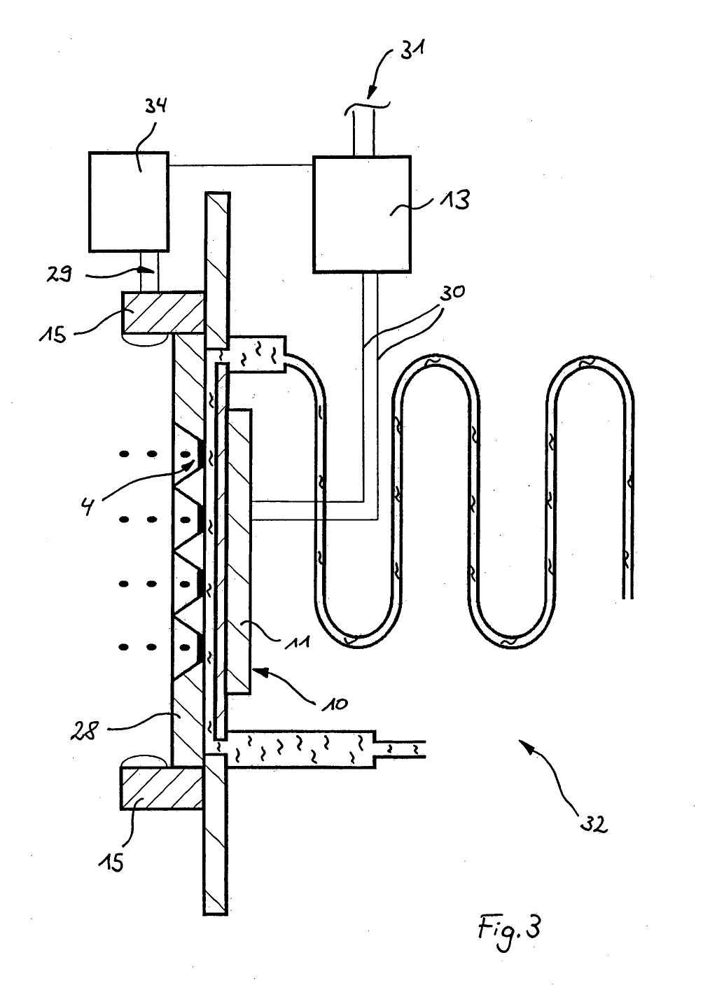

FIG. 3 shows, in a schematic representation, a dosing chamber with output function unit, storage chamber and optical measurement means,

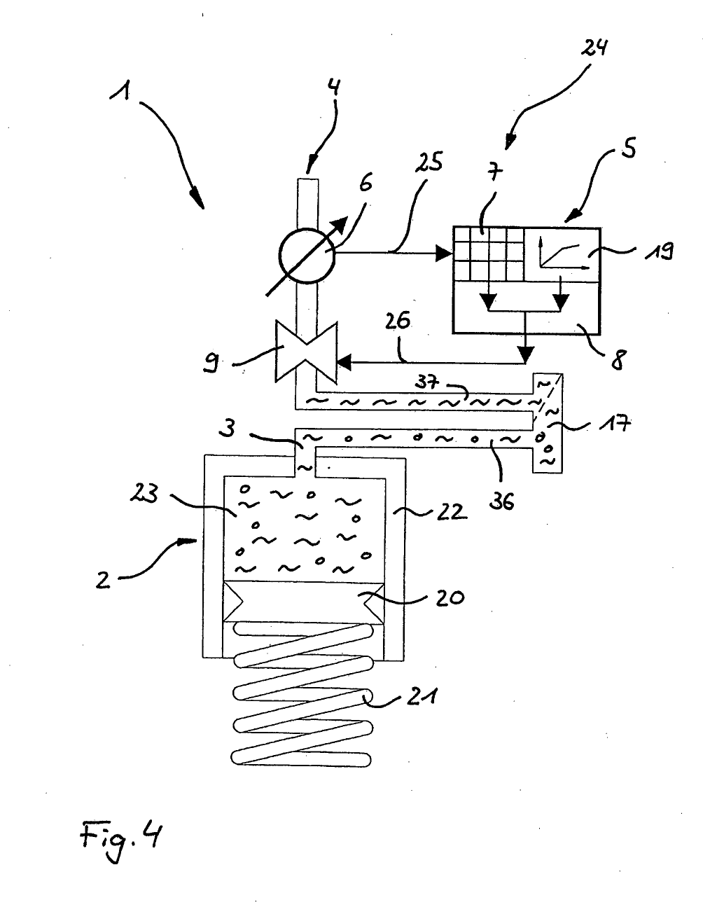

FIG. 4 shows, in a schematic representation, a dosing device with control unit and gas separator, and

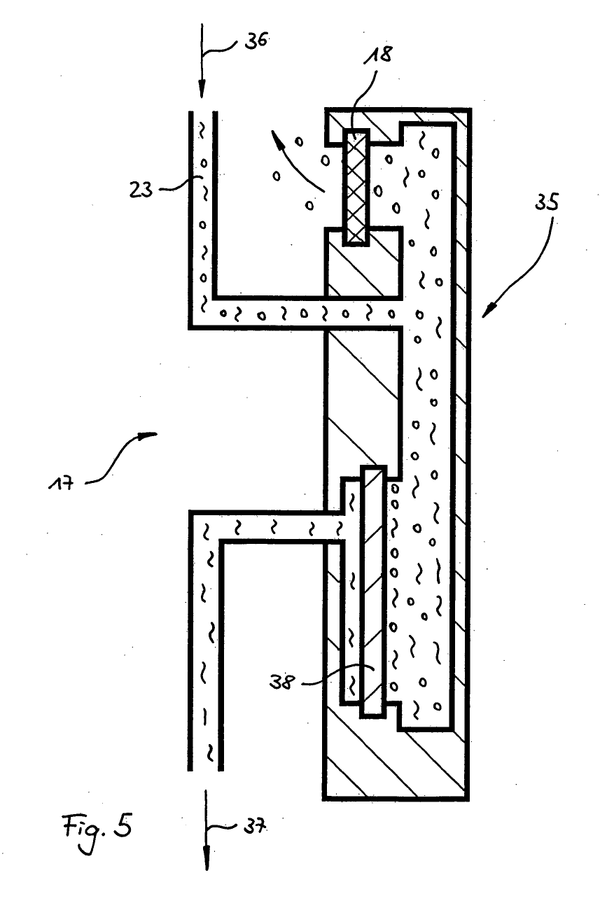

FIG. 5 shows, in a schematic representation, a gas separator with gas outlet, and

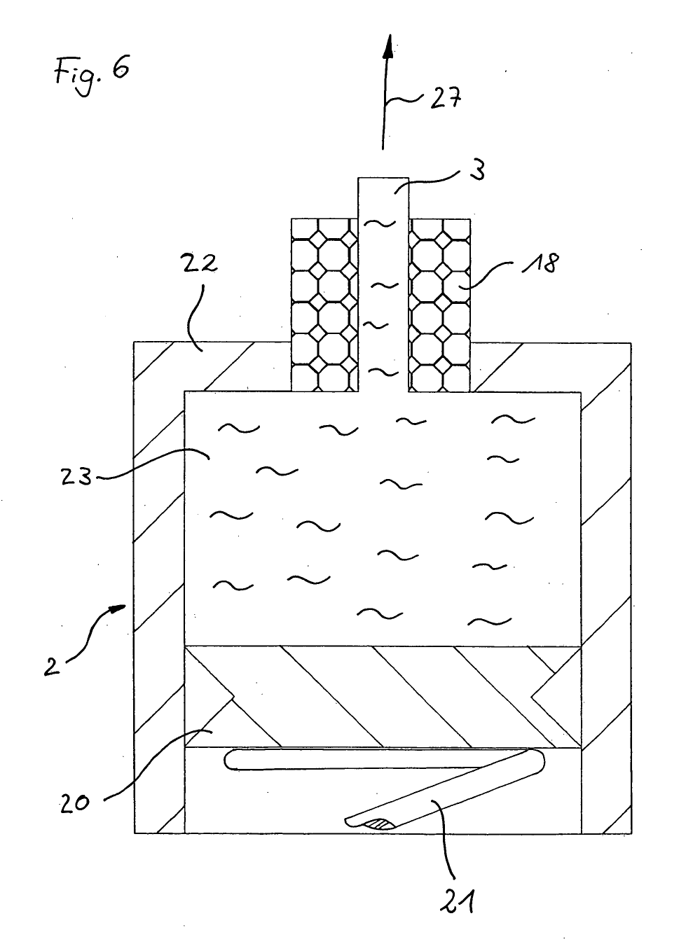

FIG. 6 shows, in a schematic representation, a medium reservoir with gas separator for a dosing device according to one of the preceding embodiments.

A dosing device 1 has a medium reservoir, designed as a pressure tank 2, and a medium control means 24. The pressure tank 2 contains, in particular in a glass container 22, a hermetically sealed quantity of liquid medium 23. The liquid medium 23 is subjected to hydrostatic pressure by a pressure piston 20 which is subjected to a pressure force by a spring means 21. The medium control means 24 has a medium channel designed as a connection tube 3, and a dispensing opening designed as a nozzle 4 (not shown in detail). An actuating means designed as a control valve 9, and a measurement means designed as a volumetric flow meter 6, are also provided on the medium control means. The volumetric flow meter 6 and the control valve 9 are coupled via a control unit designed as an integrated circuit 5. The integrated circuit 5 is supplied with an input signal 25 from the volumetric flow meter 6 and generates an output signal 26 for controlling the control valve 9. For this purpose, the input signal 25 is temporarily stored as actual value in a memory 7 of the circuit 5 and thereafter, in a comparator 8, is compared with a desired value from a desired value register 19. This comparison yields the output signal 26 which is provided to act on the control valve 9.

For dispensing medium, a dispense signal is sent via trigger means (not shown) to the integrated circuit 5 and there allows a first desired value from the desired value register 19 to enter the comparator 8. Since no dispensing operation takes place at this time, the comparator 8 detects a large difference between the desired value and the actual value determined from the input signal 25 and it triggers a corresponding output signal 26 in order to act on the control valve 9. With this output signal 26, the control valve 9 opens and thus allows the liquid medium 23 to flow out of the pressure tank 2, past the volumetric flow meter 6 and into the nozzle 4. From there, the medium passes out into the environment of the dosing device. While it is flowing past the volumetric flow meter 6, a new input signal 25 is produced and is forwarded to the memory 7 of the integrated circuit 5. In the comparator 8, this input signal is compared with the corresponding desired value from the desired value register 19 and leads to a new output signal 26. By means of this closed control loop, medium can be precisely dosed on the basis of predetermined desired values.

FIG. 2 shows an atomizing means 32 which can be coupled in downstream of the medium control means 24 and which comprises a dosing chamber, designed as an ultrasonic chamber 10, and an output function unit designed as a control device 13. A vibration unit designed as an ultrasonic oscillator 11 is assigned to the ultrasonic chamber 10. The ultrasonic oscillator 11 is fitted on a boundary surface provided as a substrate 12 and can be activated by electrical voltage via an actuating signal 30 from the control device 13. The ultrasonic chamber 10 is moreover delimited by a membrane 28 in which several dispensing openings provided as nozzles 4 are formed. The ultrasonic chamber 10 is supplied with liquid medium 23 via an inflow channel 33 and also via a storage chamber designed as a meandering reservoir 14. The meandering reservoir 14 is provided with a measurement means which is designed as a volumetric flow meter 6 and which emits a measurement signal 29 to the control device 13. From the control device 13, a synchronization signal is forwarded to the integrated circuit 5 (not shown) of the medium control means 24 in order to synchronize a function of the atomizing means 32 with the medium control means 24.

For a dispensing operation, a synchronization signal 31 is sent from the integrated circuit 5 (not shown) to the control device 13. In this way, a stream of medium is output in flow direction 27 from the medium control means (not shown) into the inflow channel 33. By means of the synchronization signal 31, an actuating signal 30 is generated in the control device 13 and acts on the ultrasonic oscillator 11, causing it to execute oscillating movements. As a result of the oscillating movements, the medium which has flowed into the ultrasonic chamber 10 is at least temporarily pressurized through a reduction in the volume of the ultrasonic chamber 10 and can be dispensed into the environment counter to the flow resistance of the nozzles 4. Since the ultrasonic oscillator 11 acts on the ultrasonic chamber 10 with in particular a periodic oscillation, the free volume in the ultrasonic chamber increases again immediately after the temporary dispensing of medium through the nozzles 4. In this way, medium can flow, in particular by capillary forces, from the meandering reservoir 14 into the ultrasonic chamber 11. No medium flows in from the inflow channel 33 because said inflow channel 33 is closed by the control valve 6 shown in FIG. 1 and FIG. 4. The flow of medium from the meandering reservoir 14 leads, according to FIG. 2, to a signal change at the volumetric flow meter 6 provided in the meandering reservoir 14. The corresponding measurement signal 29 is forwarded to the control device 13 and is there converted into a total signal for identification of a dispensed volume stream. With complete emptying of the meandering reservoir 14, the dispensing operation is terminated by the synchronization signal 31 being fed back to the integrated circuit 5 (not shown).

In the atomizing means 32 shown in FIG. 3, in contrast to the illustrative embodiment according to FIG. 2, a sensor arrangement is provided in the form of a light barrier 15 situated downstream of the membrane 28, which sensor arrangement emits a measurement signal 29 to an integration unit 34. The integration unit 34 is electrically coupled to the control device 13 and allows a measured value to be transmitted to the control device. This measured value is converted in the control device 13 to an actuating signal 30 for the ultrasonic oscillator 11 which, analogously to the embodiment in FIG. 2, can excite the ultrasonic chamber 10 so as to cause it to oscillate. The light barrier 15 determines, on the basis of a transmission value, the absolute dispensed quantity of medium, and this is forwarded to the control unit and to the integrated circuit 5 (not shown).

The embodiment of the invention shown in FIG. 4 is simply a modification of the illustrative embodiment already shown in FIG. 1, except that here a gas separator 17 is provided on the connection tube 3. The gas separator holds back gas particles from the liquid medium 23 and discharges these to the environment via a gas outlet. The liquid medium 23 freed from gas particles, by contrast, can be conveyed for onward dosing. According to FIG. 5, the gas separator is designed as a filter chamber 35 and has a medium inlet 36 and a medium outlet 37. Liquid medium 23 flows into the filter chamber 35 through the medium inlet 36, and gas particles contained in the liquid medium 23 are retained on a gas filter 38, while the liquid medium freed from gas particles can flow onwards in the direction of the medium outlet 37. The gas particles held back accumulate in the filter chamber 35 and, with increasing partial pressure, are led off into the environment via the gas outlet 18 designed as hydrophobic membrane filter. The dosing accuracy of the dosing device can thus be improved by the gas separator 17. Moreover, unnecessary consumption of energy and formation of gas bubbles during the dispensing operation in the ultrasonic chamber 10 are avoided.

In the embodiment of the pressure tank 2 serving as medium reservoir, as shown in FIG. 6, a particular addition or replacement to the gas separation arrangements shown in FIGS. 4 and 5 lies in the provision of a gas outlet 18 directly at an outlet area of the container designed in particular as a glass container 22. As in the case of the gas outlet known from FIG. 5, the gas outlet 18 according to FIG. 6 is designed as a hydrophobic membrane filter. The pressure tank is placed under static pressure via the pressure force exerted on the liquid medium 23 by the spring means 21 via the pressure piston 20. The effect of the pressure is that gas particles dissolved in the liquid medium 23 are discharged through the gas outlet 18 into the environment, so that liquid medium 23 flowing out of the pressure tank 2 in flow direction 27 and through the connection tube 3 can flow, at least substantially free from gas, into the downstream inlet channel (not shown) of the dosing device. In an embodiment not shown here, in addition to the gas outlet 18 on the pressure tank 2, it is possible, when the pressure tank 2 is connected to an ultrasonic atomizer, for a further gas separator and/or gas outlet to be provided in the immediate proximity of the ultrasonic chamber.

1. Dosing device (1) for a medium, with a medium reservoir (2), with at least one medium channel (3) for delivering the medium (23), and with at least one dispensing opening (4) for dispensing the medium (23), characterized in that the medium channel (3) is assigned a control unit (5) which comprises a measurement means (6) for detecting an actual value of a stream of medium, a memory (7) for desired values of the stream of medium, a comparator (8) for comparison between actual values and desired values, and an actuating means (9) for acting on the stream of medium as a function of evaluations performed by the comparator (8).

2. Dosing device according to claim 1, characterized in that a dosing chamber (10) provided on the medium channel (3) at least temporarily receives a quantity of liquid and is assigned the at least one dispensing opening (4), and the dosing chamber (10) is provided with a vibration unit (11) which is operatively connected at least to one boundary surface (12) of the dosing chamber (10) in order to cause this boundary surface (12) to oscillate for the purpose of a dispensing operation, and an output function unit (13) connected to the vibration unit (11) is provided for activating the vibration unit during an output period.

3. Dosing device according to claim 2, characterized in that the output function unit (13) is coupled to the control unit (5) for controlled output of medium.

4. Dosing device according to claim 1, characterized in that the measurement means (6) is provided in a storage chamber (14) assigned to the dosing chamber (10).

5. Dosing device according to claim 1, characterized in that the measurement means (6), seen in the direction of flow of the medium, is arranged downstream from the dispensing opening (4) on the dosing chamber (10).

6. Dosing device according to claim 1, characterized in that the measurement means (6) has an optical and/or capacitive and/or inductive sensor arrangement (15).

7. Dosing device according to claim 1, characterized in that the medium reservoir (2) is pressurized.

8. Dosing device according to claim 1, characterized in that a gas separator (17) is provided in the medium channel (3) and/or in the medium reservoir (2).

9. Dosing device according to claim 8, characterized in that a gas outlet (18), designed in particular as a hydrophobic membrane filter, is provided on the medium reservoir (2) and/or on the gas separator (17).
